(19) United States
(12) Patent Application Publication
(10) Pub. No.: US 2016/0002131 A1
(43) Pub. Date: Jan. 7, 2016

(54) VAPOR RECOMPRESSION

(71) Applicant: BUTAMAX ADVANCED BIOFUELS LLC, Wilmington, DE (US)

(72) Inventors: Michael Glasspool, Wilmington, DE (US); William D. Parten, Wilmington, DE (US); Joseph J. Zaher, Newark, DE (US)

(21) Appl. No.: 14/768,085

(22) PCT Filed: Feb. 14, 2014
(86) PCT No.: PCT/US2014/016398

§ 371 Date: Aug. 14, 2015

Related U.S. Application Data
(60) Provisional application No. 61/767,374, filed on Feb. 21, 2013.

(51) Int. Cl. C07C 29/80; B01D 3/00

(52) U.S. Cl. CPC C07C 29/80; B01D 3/007; B01D 3/002

(57) ABSTRACT

Provided are processes for recovering thermal energy and utilizing the recovered thermal energy as a heat source. The processes comprise distilling a butanol and water composition in a distillation unit, whereby the distillation produces a vapor comprising butanol and water at a pressure P1; compressing the vapor to a second pressure P2, wherein P2 is greater than P1; and condensing the vapor to a liquid, whereby condensing the vapor allows for the recovery of thermal energy, wherein the recovered thermal energy is used as a heat source.

### CROSS REFERENCE TO RELATED APPLICATION

This application claims benefit of priority from U.S. Provisional Application No. 61/767,374, filed Feb. 21, 2013, which is hereby incorporated by reference in its entirety.

### FIELD OF INVENTION

The present invention relates to processes for recovering thermal energy and utilizing the recovered thermal energy as a heat source. More specifically, the invention relates to processes for recovering thermal energy and utilizing the recovered thermal energy as a heat source in a butanol production plant to reduce energy requirements.

### BACKGROUND

Butanol is an important industrial chemical, useful as a fuel additive, as a feedstock chemical in the plastics industry, and as a food grade extractant in the food and flavor industry. Accordingly, there is a high demand for butanol, as well as for efficient production methods. One production method which has the potential to reduce environmental impact includes the production of butanol utilizing fermentation by recombinant microorganisms. For large-scale production of butanol utilizing fermentation by recombinant microorganisms, production improvements can improve efficiency and economics. A reduction in the total plant energy consumption for the process is one such desirable improvement.

The present invention satisfies the need to provide an alternative source of energy to reduce the energy requirements for the distillation of a product alcohol (e.g., butanol) produced by a fermentation process.

### SUMMARY

Provided herein are processes for recovering thermal energy and utilizing the recovered thermal energy as a heat source. The processes comprise (a) distilling a butanol and water composition in a distillation unit, whereby the distillation produces a vapor comprising butanol and water at a first pressure P1; (b) compressing the vapor to a second pressure P2, wherein P2 is greater than P1; and (c) condensing the vapor to a liquid, whereby condensing the vapor allows for the recovery of thermal energy, wherein the recovered thermal energy is used as a heat source. In certain embodiments, the butanol can be bio-produced.

Also provided are processes comprising (a) providing a fermentation medium comprising a recombinant microorganism comprising a biosynthetic butanol pathway, whereby the recombinant microorganism produces butanol; (b) contacting the butanol with an extractant; (c) distilling the butanol and extractant in a distillation unit, whereby the distillation produces a vapor comprising butanol and water, wherein the vapor is produced at a first pressure P1; (d) compressing the vapor to a second pressure P2, wherein P2 is greater than P1; and (e) condensing the vapor to a liquid, whereby condensing the vapor allows for the recovery of thermal energy, wherein the recovered thermal energy is used as a heat source.

Optionally, the first pressure P1 is in a range from about 0.1 atmospheres (atm) to about 2 atm. Optionally, the first pressure P1 is about 0.45 atm. Optionally, the second pressure P2 is in a range from about 0.11 atm to about 10 atm. Optionally, the second pressure P2 is about 0.90 atm. In certain embodiments, the ratio of P2 to P1 is in a range from about 1.1 to about 5.0. Optionally, the ratio of P2 to P1 is in a range from about 1.5 to about 3.5. Optionally, the ratio of P2 to P1 is in a range from about 1.8 to about 2.2.

Optionally, the vapor is compressed by a compressor selected from the group consisting of a centrifugal compressor, an axial compressor, a liquid ring compressor, a reciprocating compressor, a rotary screw compressor, and a diaphragm compressor. In certain embodiments, the vapor is compressed by a centrifugal compressor.

The distillation unit can, for example, comprise at least one distillation column. Optionally, the at least one distillation column can comprise at least one reboiler. In certain embodiments, the recovered thermal energy can be used to heat the at least one reboiler. Optionally, the at least one distillation column can comprise a feed, wherein the feed comprises a composition comprising butanol and water. Optionally, the feed can further comprise an extractant. In certain embodiments, the recovered thermal energy can be used to heat the feed.

The recombinant microorganism can comprise a butanol biosynthetic pathway selected from the group consisting of (a) a 1-butanol biosynthetic pathway; (b) a 2-butanol biosynthetic pathway; and (c) an isobutanol biosynthetic pathway.

Optionally, the 1-butanol biosynthetic pathway comprises at least one polypeptide that performs one of the following substrate to product conversions: (a) acetyl-CoA to acetoacetyl-CoA, as catalyzed by acetyl-CoA acetyltransferase; (b) acetoacetyl-CoA to 3-hydroxybutyryl-CoA, as catalyzed by 3-hydroxybutyryl-CoA dehydrogenase; (c) 3-hydroxybutyryl-CoA to crotonyl-CoA, as catalyzed by crotonase; (d) crotonyl-CoA to butyryl-CoA, as catalyzed by butyryl-CoA dehydrogenase; (e) butyryl-CoA to butyraldehyde, as catalyzed by butyraldehyde dehydrogenase; and (f) butyraldehyde to 1-butanol, as catalyzed by 1-butanol dehydrogenase.

Optionally, the 2-butanol biosynthetic pathway comprises at least one polypeptide that performs one of the following substrate to product conversions: (a) pyruvate to alpha-acetolactate, as catalyzed by acetolactate synthase; (b) alpha-acetolactate to acetoin, as catalyzed by acetolactate decarboxylase; (c) acetoin to 2,3-butanediol, as catalyzed by butanediol dehydrogenase; (d) 2,3-butanediol to 2-butanone, as catalyzed by butanediol dehydratase; and (e) 2-butanone to 2-butanol, as catalyzed by 2-butanol dehydrogenase.

Optionally, the isobutanol biosynthetic pathway comprises at least one polypeptide that performs one of the following substrate to product conversions: (a) pyruvate to acetolactate, as catalyzed by acetolactate synthase; (b) acetolactate to 2,3-dihydroxyisovalerate, as catalyzed by ketol-acid reductoisomerase (c) 2,3-dihydroxyisovalerate to α-ketoisovalerate, as catalyzed by dihydroxyacid dehydratase; (d) α-ketoisovalerate to isobutyraldehyde, as catalyzed by a branched chain keto acid decarboxylase; and (e) isobutyraldehyde to isobutanol, as catalyzed by branched-chain alcohol dehydrogenase.

The extractant can, for example, comprise C₇ to C₂₂ fatty alcohols, esters of C₇ to C₂₂ fatty acids, C₇ to C₂₂ fatty aldehydes, C₇ to C₂₂ fatty amides, or mixtures thereof. In certain embodiments, the extractant can comprise oleyl alcohol, behenyl alcohol, cetyl alcohol, lauryl alcohol, myristyl alcohol, stearyl alcohol, oleic acid, lauric acid, myristic acid, stearic acid, octanoic acid, decanoic acid, undecanoic acid, methyl myristate, methyl oleate, 1-nonanol, 1-decanol, 2-undecanol, 1-nonanal, 1-undecanol, undecanal, lauric aldehyde, 2-methylundecanal, oleamide, linoleamide, palmitamide, stearylamide, 2-ethyl-1-hexanol, 2-hexyl-1-decanol, 2-octyl-1-dodecanol, or mixtures thereof. In certain embodiments, the extractant comprises fatty acids derived from a plant source. The plant source, can, for example, be selected from corn, sugar cane, wheat, soy, barley, rice, switchgrass, grain, sorghum, whey, or mixtures thereof.

### FIGURE DESCRIPTION

FIG. 1 shows a schematic of a distillation column of a distillation unit employing vapor recompression for providing heat energy to a reboiler for self-heated distillation.

FIG. 2 shows a schematic of a distillation column of a distillation unit employing vapor recompression for providing heat energy to a feed in a second distillation column.

FIG. 3 shows a schematic of a distillation column of a distillation unit employing vapor recompression for providing heat energy to a reboiler of a second distillation column.

### DETAILED DESCRIPTION

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

In order to further define this invention, the following terms and definitions are herein provided.

As used herein, the terms “comprises,” “comprising,” “includes,” “including,” “has,” “having,” “contains,” or “containing,” or any variation thereof, will be understood to imply the inclusion of a stated integer or group of integers but not the exclusion of any other integer or group of integers. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein, is a non-limiting term and is not intended to refer to any single embodiment of the particular invention but encompasses all possible embodiments as described in the application.

As used herein, the term “about” modifying the quantity of an ingredient or reactant of the invention employed refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about,” the claims include equivalents to the quantities. In one embodiment, the term “about” means within 10% of the reported numerical value, alternatively within 5% of the reported numerical value.

“Biomass” as used herein, refers to a natural product comprising hydrolysable polysaccharides that provide fermentable sugars, including any sugars and starch derived from natural resources such as corn, sugar cane, wheat, cellulosic or lignocellulosic material and materials comprising cellulose, hemicellulose, lignin, starch, oligosaccharides, disaccharides and/or monosaccharides, and mixtures thereof. Biomass may also comprise additional components, such as protein and/or lipids. Biomass may be derived from a single source, or biomass can comprise a mixture derived from more than one source; for example biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, waste sugars, wood and forestry waste. Examples of biomass include, but are not limited to, corn grain, corn cobs, crop residues such as corn husks, corn stover, grasses, wheat, rye, wheat straw, barley, barley straw, hay, rice straw, switchgrass, waste paper, sugar cane bagasse, sorghum, soy, whey, whey permeate, components obtained from milling of grains, trees, branches, roots, leaves, wood chips, sawdust, shrubs and bushes, vegetables, fruits, flowers, animal manure, and mixtures thereof. For example, mash, juice, molasses, or hydrolysate may be formed from biomass by any processing known in the art for processing the biomass for purposes of fermentation, such as by milling, treating and/or liquefying and comprises fermentable sugar and may comprises an amount of water. For example, corn may be processed via wet mill or dry mill and subsequently liquefied to produce mash. Cellulosic and/or lignocellulosic biomass may be processed to obtain a hydrolysate containing fermentable sugars by any method known to one skilled in the art (see, e.g., U.S. Patent Application Publication No. 2007/0031918, which is herein incorporated by reference). Enzymatic saccharification of cellulosic and/or lignocellulosic biomass makes use of an enzyme consortium for breaking down cellulose and hemicellulose to produce a hydrolysate containing sugars including glucose, xylose, and arabinose. Saccharification enzymes suitable for cellulosic and/or lignocellulosic biomass are reviewed in Lynd et al., Microbiol. Mol. Biol. Rev. 66:506-77 (2002).

“Biomass yield” as used herein, refers to the ratio of microorganism biomass produced (i.e., cell biomass production) to carbon substrate consumed.

“Biofuel” or “biofuel product” as used herein, refers to a fuel derived from a biological process, for example, but not limited to, fermentation.

“Butanol” as used herein, refers to the butanol isomers 1-butanol (1-BuOH), 2-butanol (2-BuOH), tert-butanol (t-BuOH), and/or isobutanol (iBuOH or i-BuOH, also known as 2-methyl-1-propanol), either individually or as mixtures thereof. From time to time, as used herein the terms “biobutanol” and “bio-produced butanol” may be used synonymously with “butanol.”

Uses for butanol can include, but are not limited to, fuels (e.g., biofuels), a fuel additive, an alcohol used for the production of esters that can be used as diesel or biodiesel fuel, as a chemical in the plastics industry, an ingredient in formulated products such as cosmetics, and a chemical intermediate. Butanol may also be used as a solvent for paints, coatings, varnishes, resins, gums, dyes, fats, waxes, resins, shellac, rubbers, and alkaloids.

As used herein, the term “bio-produced” means that the molecule (e.g., butanol) is produced from a renewable source (e.g., the molecule can be produced during a fermentation process from a renewable feedstock). Thus, for example, bio-produced isobutanol can be isobutanol produced by a fermentation process from a renewable feedstock. Molecules produced from a renewable source can further be defined by the ¹⁴C/¹²C isotope ratio. A ¹⁴C/¹²C isotope ratio in range of from 1:0 to greater than 0:1 indicates a bio-produced molecule, whereas a ratio of 0:1 indicates that the molecule is fossil derived.

“Product alcohol” as used herein, refers to any alcohol that can be produced by a microorganism in a fermentation process that utilizes biomass as a source of fermentable carbon substrate. Product alcohols include, but are not limited to, C₁ to C₈ alkyl alcohols, and mixtures thereof. In some embodiments, the product alcohols are C₂ to C₈ alkyl alcohols. In other embodiments, the product alcohols are C₂ to C₅ alkyl alcohols. It will be appreciated that C₁ to C₈ alkyl alcohols include, but are not limited to, methanol, ethanol, propanol, butanol, pentanol, and mixtures thereof. Likewise C₂ to C₈ alkyl alcohols include, but are not limited to, ethanol, propanol, butanol, and pentanol. “Alcohol” is also used herein with reference to a product alcohol.

The term “effective titer” as used herein, refers to the total amount of a particular alcohol (e.g., butanol) produced by fermentation or alcohol equivalent of the alcohol ester produced by alcohol esterification per liter of fermentation medium. For example, the effective titer of butanol in a unit of volume of a fermentation includes: (i) the amount of butanol in the fermentation medium; (ii) the amount of butanol recovered from the organic extractant; (iii) the amount of butanol recovered from the gas phase, if gas stripping is used; and (iv) the alcohol equivalent of the butyl ester in either the organic or aqueous phase.

The term “effective rate” as used herein, is the effective titer divided by the fermentation time.

The term “effective yield” as used herein, is the total grams of product alcohol produced per gram of glucose consumed.

“In Situ Product Removal” (ISPR) as used herein, means the selective removal of a fermentation product from a biological process such as fermentation to control the product concentration as the product is produced.

“Fermentable carbon source” or “fermentable carbon substrate” as used herein, means a carbon source capable of being metabolized by the microorganisms disclosed herein for the production of fermentative alcohol. Suitable fermentable carbon sources include, but are not limited to, monosaccharides such as glucose or fructose; disaccharides such as lactose or sucrose; oligosaccharides; polysaccharides such as starch or cellulose; C5 sugars such as xylose and arabinose; one carbon substrates including methane; amino acids; and mixtures thereof.

“Feedstock” as used herein, means a feed in a fermentation process, the feed containing a fermentable carbon source with or without undissolved solids, and where applicable, the feed containing the fermentable carbon source before or after the fermentable carbon source has been liberated from starch or obtained from the breakdown of complex sugars by further processing such as by liquefaction, saccharification, or other process. Feedstock includes or is derived from a biomass. Suitable feedstocks include, but are not limited to, rye, wheat, corn, corn mash, cane, cane mash, barley, cellulosic material, lignocellulosic material, or mixtures thereof. Where reference is made to “feedstock oil,” it will be appreciated that the term encompasses the oil produced from a given feedstock.

“Sugar” as used herein, refers to oligosaccharides, disaccharides, monosaccharides, and/or mixtures thereof. The term “saccharide” also includes carbohydrates including starches, dextrans, glycogens, cellulose, pentosans, as well as sugars.

“Fermentable sugar” as used herein, refers to one or more sugars capable of being metabolized by the microorganisms disclosed herein for the production of fermentative alcohol.

“Undissolved solids” as used herein, means non-fermentable portions of feedstock, for example, germ, fiber, and gluten. For example, the non-fermentable portions of feedstock include the portion of feedstock that remains as solids and can absorb liquid from the fermentation broth.

“Fermentation medium” as used herein, means the mixture of water, sugars, dissolved solids, optionally microorganisms producing alcohol, product alcohol, and all other constituents of the material in which product alcohol is made by the reaction of sugars to alcohol, water, and carbon dioxide (CO₂) by the microorganisms present. From time to time, as used herein the term “fermentation broth” and “fermented mixture” can be used synonymously with “fermentation medium.”

The term “biphasic fermentation medium” as used herein, refers to a two-phase growth medium comprising a fermentation medium (i.e., aqueous phase) and a suitable amount of a water immiscible organic extractant.

“Fermentation vessel” as used herein, means the vessel in which the fermentation reaction is carried out whereby product alcohol such as butanol is made from sugars. “Fermentor” may be used herein interchangeable with “fermentation vessel.”

The terms “separation” as used herein is synonymous with “recovery,” “recovering,” or variants thereof and refers to removing a chemical compound from an initial mixture to obtain the compound in greater purity or at a higher concentration than the purity or concentration of the compound in the initial mixture.

“Extractant” as used herein, means a solvent used to remove or separate a product alcohol such as butanol. From time to time, as used herein the term “solvent” may be used synonymously with “extractant.” For the processes described herein, extractants are water immiscible.

“Water immiscible” or “insoluble” as used herein, refers to a chemical component such as an extractant or solvent, which is incapable of mixing with an aqueous solution such as a fermentation broth, in such a manner as to form one liquid phase.

The term “aqueous phase” as used herein, refers to the aqueous phase of a biphasic mixture obtained by contacting a fermentation broth with a water-immiscible organic extractant. In an embodiment of a process described herein that includes fermentative extraction, the term “fermentation broth” then specifically refers to the aqueous phase in biphasic fermentative extraction, and the terms “solvent-poor phase” may be used synonymously with “aqueous phase” and “fermentation broth.” In addition, undissolved solids (e.g., grain solids) can be present in the fermentation broth, such that the biphasic mixture includes the undissolved solids which are primarily dispersed in the aqueous phase.

The term “aqueous phase titer” as used herein, refers to the concentration of product alcohol (e.g., butanol) in the fermentation broth.

The term “organic phase” as used herein, refers to the non-aqueous phase of a biphasic mixture obtained by contacting a fermentation broth with a water-immiscible organic extractant. From time to time, as used herein the terms “solvent-rich phase” may be used synonymously with “organic phase.”

The term “distillation unit” as used herein, refers to the one or more distillation columns that are used to separate a mixture of compounds (e.g., butanol from water) in the processes described herein. Distillation columns can serve different functions. In certain embodiments, a distillation column can serve to separate a product alcohol (e.g., butanol) from the fermentation broth and can be referred to as the “beer column.” In certain embodiments, a distillation column can serve to separate a product alcohol (e.g., butanol) from an extractant, clean the extractant for recycle and can be referred to as the “extractant column.” In certain embodiments, a distillation column can separate water from a product alcohol (e.g., butanol) and can be referred to as the “rectifying column.” In certain embodiments, a distillation column can serve to clean process recycle water, which can occur in both a “beer column” and a “side stripping column.”

Unless otherwise indicated, the term “separate” does not require any particular degree of separation. By way of example, a butanol and water composition of about 7:93 (v/v) can be separated into a butanol/water stream of about 50:50 and a substantially pure water stream. By way of another example, a butanol and water composition of about 85:15 (v/v) can be separated into a butanol/water stream of about 73:27 and a substantially pure butanol stream. By way of another example, a butanol and water composition of about 74:26 (v/v) can be separated into a butanol/water stream of about 66:34 and a water/butanol stream of about 24:76. One of skill in the art will understand what is meant by the term separate. Likewise, it will be understood that the use of a distillation column can result in a substantially complete separation of the mixture/composition.

The term “stripping” as used herein, refers to the action of transferring all or part of a volatile component from a liquid stream to a gaseous stream.

The term “stripping section” as used herein, refers to the part of the contacting device in which the stripping operation takes place. A “stripping section” can, for example, be in a stripping column in a distillation unit.

The term “rectifying” as used herein, refers to the action of transferring all or a portion of a condensable component from a gaseous stream into a liquid stream in order to separate and purify lower boiling point components from higher boiling point components.

The term “rectifying section” as used herein, refers to the section of the distillation column above the feed point, i.e., the trays or packing material located above the point in the column where the feed stream enters, where the rectifying operations takes place. A “rectifying section” can, for example, be in a rectifying column in a distillation unit.

The term “condensing temperature” as used herein, refers to the temperature at which the resultant condensed liquid is produced. The condensing temperature of a vapor composition can be between the dew point of the vapor composition and bubble point of the resultant liquid composition.

The term “fatty acid” as used herein, refers to a carboxylic acid (e.g., aliphatic monocarboxylic acid) having C₄ to C₂₈ carbon atoms (most commonly C₁₂ to C₂₄ carbon atoms), which is either saturated or unsaturated. Fatty acids may also be branched or unbranched. Fatty acids may be derived from, or contained in esterified form, in an animal or vegetable fat, oil, or wax. Fatty acids may occur naturally in the form of glycerides in fats and fatty oils or may be obtained by hydrolysis of fats or by synthesis. The term fatty acid may describe a single chemical species or a mixture of fatty acids. In addition, the term fatty acid also encompasses free fatty acids.

The term “fatty alcohol” as used herein, refers to an alcohol having an aliphatic chain of C₄ to C₂₂ carbon atoms, which is either saturated or unsaturated.

The term “fatty aldehyde” as used herein, refers to an aldehyde having an aliphatic chain of C₄ to C₂₂ carbon atoms, which is either saturated or unsaturated.

The term “fatty amide” as used herein, refers to an amide having a long, aliphatic chain of C₄ to C₂₂ carbon atoms, which is either saturated or unsaturated.

The term “fatty ester” as used herein, refers to an ester having a long aliphatic chain of C₄ to C₂₂ carbon atoms, which is either saturated or unsaturated.

The term “carboxylic acid” as used herein, refers to any organic compound with the general chemical formula —COOH in which a carbon atom is bonded to an oxygen atom by a double bond to make a carbonyl group (—C═O) and to a hydroxyl group (—OH) by a single bond. A carboxylic acid may be in the form of the protonated carboxylic acid, in the form of a salt of a carboxylic acid (e.g., an ammonium, sodium, or potassium salt), or as a mixture of protonated carboxylic acid and salt of a carboxylic acid. The term carboxylic acid may describe a single chemical species (e.g., oleic acid) or a mixture of carboxylic acids as can be produced, for example, by the hydrolysis of biomass-derived fatty acid esters or triglycerides, diglycerides, monoglycerides, and phospholipids.

“Portion” as used herein, includes a part of a whole or the whole. For example, a portion of fermentation broth includes a part of the fermentation broth as well as the whole (or all) the fermentation broth.

“Partition coefficient” refers to the ratio of the concentration of a compound in the two phases of a mixture of two immiscible solvents at equilibrium. A partition coefficient is a measure of the differential solubility of a compound between two immiscible solvents. Partition coefficient, as used herein, is synonymous with the term distribution coefficient.

As used herein, the term “recombinant microorganism” refers to microorganisms such as bacteria or yeast that are modified by use of recombinant DNA techniques. The recombinant microorganism can, for example, be engineered to express a metabolic pathway and/or the recombinant microorganism can be engineered to reduce or eliminate undesired products and/or increase the efficiency of the desired metabolite. As an example, a recombinant microorganism can be made by engineering a host cell to comprise a biosynthetic pathway such as a biosynthetic pathway to produce an alcohol such as butanol.

A recombinant host cell comprising an “engineered alcohol production pathway” (such as an engineered butanol or isobutanol production pathway) refers to a host cell containing a modified pathway that produces alcohol in a manner different than that normally present in the host cell. Such differences include production of an alcohol not typically produced by the host cell, or increased or more efficient production.

The term “heterologous biosynthetic pathway” as used herein refers to an enzyme pathway to produce a product in which at least one of the enzymes is not endogenous to the host cell containing the biosynthetic pathway.

The term “butanol biosynthetic pathway” as used herein refers to the enzymatic pathway to produce 1-butanol, 2-butanol, or isobutanol.

The term “1-butanol biosynthetic pathway” refers to the enzymatic pathway to produce 1-butanol. A “1-butanol biosynthetic pathway” can refer to an enzyme pathway to produce 1-butanol from acetyl-coenzyme A (acetyl-CoA). For example, 1-butanol biosynthetic pathways are disclosed in U.S. Patent Application Publication No. 2008/0182308 and International Publication No. WO 2007/041269, which are incorporated by reference herein in their entireties.

The term “2-butanol biosynthetic pathway” refers to the enzymatic pathway to produce 2-butanol. A “2-butanol biosynthetic pathway” can refer to an enzyme pathway to produce 2-butanol from pyruvate. For example, 2-butanol biosynthetic pathways are disclosed in U.S. Pat. No. 8,206,970; U.S. Patent Application Publication No. 2007/0292927; International Publication Nos. WO 2007/130518 and WO 2007/130521, which are incorporated by reference herein in their entireties.

The term “isobutanol biosynthetic pathway” refers to the enzymatic pathway to produce isobutanol. An “isobutanol biosynthetic pathway” can refer to an enzyme pathway to produce isobutanol from pyruvate. For example, isobutanol biosynthetic pathways are disclosed in U.S. Pat. No. 7,851,188; U.S. Application Publication No. 2007/0092957; and International Publication No. WO 2007/050671, which are incorporated by reference herein in their entireties. From time to time “isobutanol biosynthetic pathway” is used synonymously with “isobutanol production pathway”.

### Vapor Recompression

To produce product alcohols (e.g., butanol) utilizing fermentation by recombinant microorganisms at large scale, the process is desirably efficient and economical such that the product alcohol is affordable. Streamlining the process to efficiently and economically produce the product alcohol can involve reducing the total plant energy consumption needed for the process. For example for the mass production of butanol, the energy source used for the distillation of the butanol and water vapors out of a mixture of the butanol, water, and organic solvent should be amenable to an ethanol plant retrofit. The energy for this distillation can be taken by injecting water vapor produced in the evaporators directly into the column; however, the energy requirements for distillation can exceed that which is provided by the evaporator heat exchangers. The present invention satisfies the need to provide an additional source of energy to reduce the energy requirements for the distillation of a product alcohol (e.g., butanol) produced by a fermentation process.

Provided herein are processes for recovering thermal energy and utilizing the recovered thermal energy as a heat source. The processes comprise (a) distilling a butanol and water composition in a distillation unit, whereby the distillation produces a vapor comprising butanol and water at a first pressure P1; (b) compressing the vapor to a second pressure P2, wherein P2 is greater than P1; and (c) condensing the vapor to a liquid, whereby condensing the vapor allows for the recovery of thermal energy, wherein the recovered thermal energy is used as a heat source.

Also provided are processes comprising (a) providing a fermentation medium comprising a recombinant microorganism comprising a biosynthetic butanol pathway, whereby the recombinant microorganism produces butanol; (b) contacting the butanol with an extractant; (c) distilling the butanol and extractant in a distillation unit, whereby the distillation produces a vapor comprising butanol and water, wherein the vapor is produced at a first pressure P1; (d) compressing the vapor to a second pressure P2, wherein P2 is greater than P1; and (e) condensing the vapor to a liquid, whereby condensing the vapor allows for the recovery of thermal energy, wherein the recovered thermal energy is used as a heat source.

Optionally, the first pressure P1 is in a range from about 0.1 atmospheres (atm) to about 2 atm. Optionally, the first pressure P1 is about 0.45 atm. Optionally, the second pressure P2 is in a range from about 0.11 atm to about 10 atm. Optionally, the second pressure P2 is about 0.90 atm. In certain embodiments, the ratio of P2 to P1 is in a range from about 1.1 to about 5.0. Optionally, the ratio of P2 to P1 is in a range from about 1.5 to about 3.5. Optionally, the ratio of P2 to P1 is in a range from about 1.8 to about 2.2.

Vapor produced a first pressure, P1, can be at a first temperature T1. Compressing the vapor to a second pressure P2 can increase the vapor temperature to a second temperature T2, wherein the increase in temperature is dependent on the increase in pressure. The second pressure, P2, can be chosen based on the desired condensing temperature, T3, such that the condensing temperature is greater than the resultant temperature, T4, of the heated stream. In certain embodiments, T3 can be greater than T4 by a range of about 1° C. to about 40° C. In certain embodiments, T3 can be greater than T4 by a range of about 3° C. to about 30° C. In certain embodiments, T3 can be greater than T4 by a range of about 4° C. to about 20° C. In certain embodiments, T3 can be greater than T4 by a range of about 5° C. to about 10° C. In certain embodiments, T3 can be greater than T4 by about 1° C., 2° C., 3° C., 4° C., 5° C., 6° C., 7° C., 8° C., 9° C., 10° C., 20° C., 30° C., or 40° C.

Optionally, the vapor is compressed by a compressor selected from the group consisting of a centrifugal compressor, an axial compressor, a liquid ring compressor, a reciprocating compressor, a rotary screw compressor, and a diaphragm compressor. In certain embodiments, the vapor is compressed by a centrifugal compressor. Compressors are known in the art, see, e.g., API Standard 617, 7^{th} Edition, “Axial and Centrifugal Compressors and Expander-compressors for Petroleum, Chemical and Gar Industry Services,” July 2002. Compression of the vapor by a compressor utilizes electrical energy to recover the thermal energy. The amount of electrical energy utilized may be less than the potential thermal energy that can be recovered. This may allow for the reduction in the total plant energy requirement for the production of the product alcohol.

The distillation unit can, for example, comprise at least one distillation column. Optionally, the at least one distillation column can comprise at least one reboiler. In certain embodiments, the recovered thermal energy can be used to heat the at least one reboiler. Optionally, the at least one distillation column can comprise a feed, wherein the feed comprises a composition comprising butanol and water. Optionally, the feed can further comprise an extractant. In certain embodiments, the recovered thermal energy can be used to pre-heat the feed prior to entry to the distillation column.

The extractant can, for example, comprise C₇ to C₂₂ fatty alcohols, esters of C₇ to C₂₂ fatty acids, C₇ to C₂₂ fatty aldehydes, C₇ to C₂₂ fatty amides, or mixtures thereof. In certain embodiments, the extractant can comprise oleyl alcohol, behenyl alcohol, cetyl alcohol, lauryl alcohol, myristyl alcohol, stearyl alcohol, oleic acid, lauric acid, myristic acid, stearic acid, octanoic acid, decanoic acid, undecanoic acid, methyl myristate, methyl oleate, 1-nonanol, 1-decanol, 2-undecanol, 1-nonanal, 1-undecanol, undecanal, lauric aldehyde, 2-methylundecanal, oleamide, linoleamide, palmitamide, stearylamide, 2-ethyl-1-hexanol, 2-hexyl-1-decanol, 2-octyl-1-dodecanol, or mixtures thereof. In certain embodiments, the extractant comprises fatty acids derived from a plant source. The plant source, can, for example, be selected from corn, sugar cane, wheat, soy, barley, rice, switchgrass, grain, sorghum, whey, or mixtures thereof.

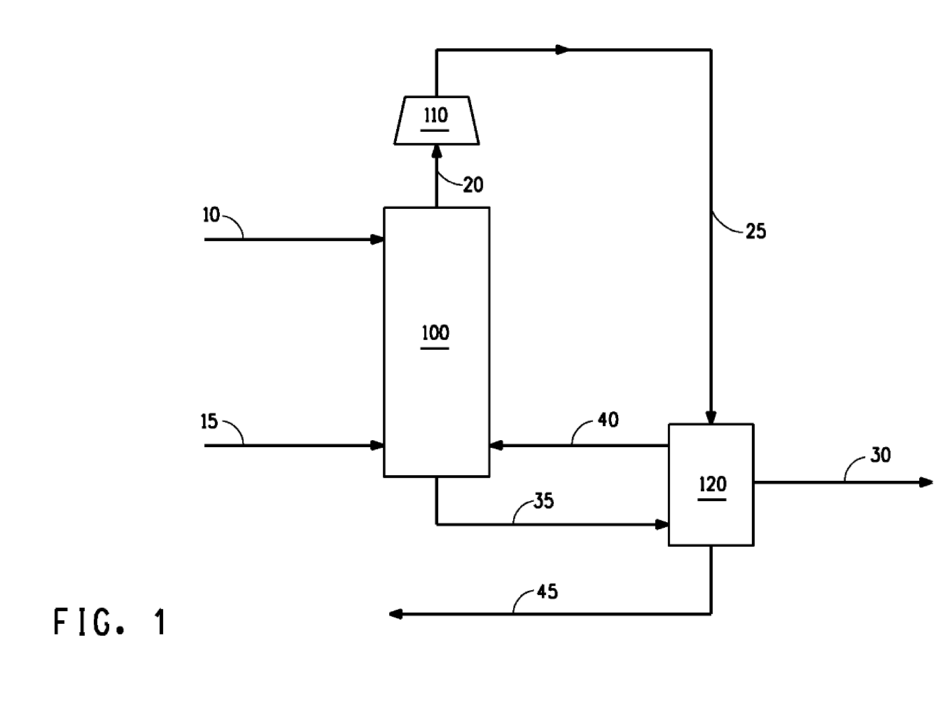

As illustrated in FIG. 1, in certain embodiments vapor recompression may be used to self-heat a column used for the distillation of a mixture comprising butanol and water. In FIG. 1, stream 10 may comprise a liquid composition feed comprising butanol and water. Stream 10 may be top fed into a distillation column 100, which can receive heat energy via the addition of vapor stream 15 comprising water. The distillation column may comprise a reboiler 120. The overheads product of the distillation, stream 20, may be a mixture comprising butanol and water at a first temperature T₁ and a first pressure P₁. The overheads stream may be compressed using a compressor 110, which can be powered by electricity. The product of the compression may be a butanol and water vapor stream 25, at a second temperature T₂ and second pressure P₂, operated such that P₂ is greater than P₁. In this embodiment the vapor product, stream 25, may be directed to the column reboiler where it can be condensed at a third temperature T₃, and the energy released from condensing the vapors may be sufficient to boil the liquid stream 35 entering the column reboiler to produce a vapor stream 40 comprising butanol and water at a fourth temperature T₄. The compression pressure, P₂, may be selected such that the vapor condensing temperature T₃ is greater than the operating temperature T₄ at the bottom of the column. The column bottom product, stream 45, can be a liquid stream comprising water, wherein the water is substantially free of butanol.

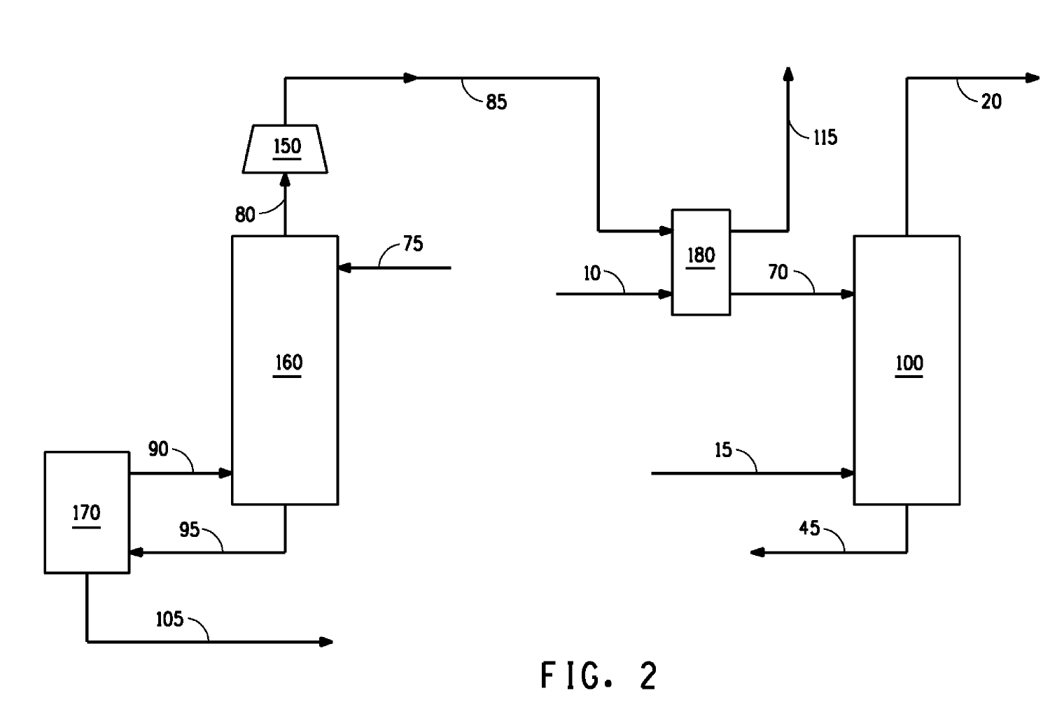

As illustrated in FIG. 2, in certain embodiments vapor recompression from a distillation column may be used to provide heat energy into a feed in a second distillation column. In FIG. 2, a liquid stream 75 comprising butanol and water may be top fed to a distillation column 160 comprising a reboiler 170. Heat may be provided to the column reboiler to vaporize the liquid stream 95 to form vapor steam 90 comprising butanol and water. The bottoms product 105 may be a liquid stream comprising butanol, wherein the butanol can be substantially free of water. The overheads vapor product 80 may be a mixture comprising butanol and water at a first temperature T₁ and first pressure P₁. This vapor stream may be compressed using a compressor 150, which may be powered electrically. The product of the compression may comprise a butanol and water vapor stream 85 at a second temperature T₂ and second pressure P₂. The compressor may be operated such that P₂ is greater than P₁. In this embodiment, the resulting vapor stream 85 may be condensed across a heat exchanger 180 to produce a condensed liquid product stream 115 at a third temperature T₃. The heat released from condensing vapor stream 85 may be used to preheat the liquid feed stream 10 to distillation column 100. Stream 10 may be a mixture comprising butanol and water and may be preheated to form stream 70 at a fourth temperature T₄, operated such that T₄ is at least 5° C. less than T₃. Thermal energy transferred in heat exchanger 180 may supply at least a portion of the energy required for the separation performed in distillation column 100. Distillation column 100 may be fed by stream 70 and stream 15, which may be a vapor stream comprising water. Distillation may produce a bottoms liquid stream 45. Distillation may also produce an overhead vapor stream 20, wherein the vapor stream may comprise butanol and water.

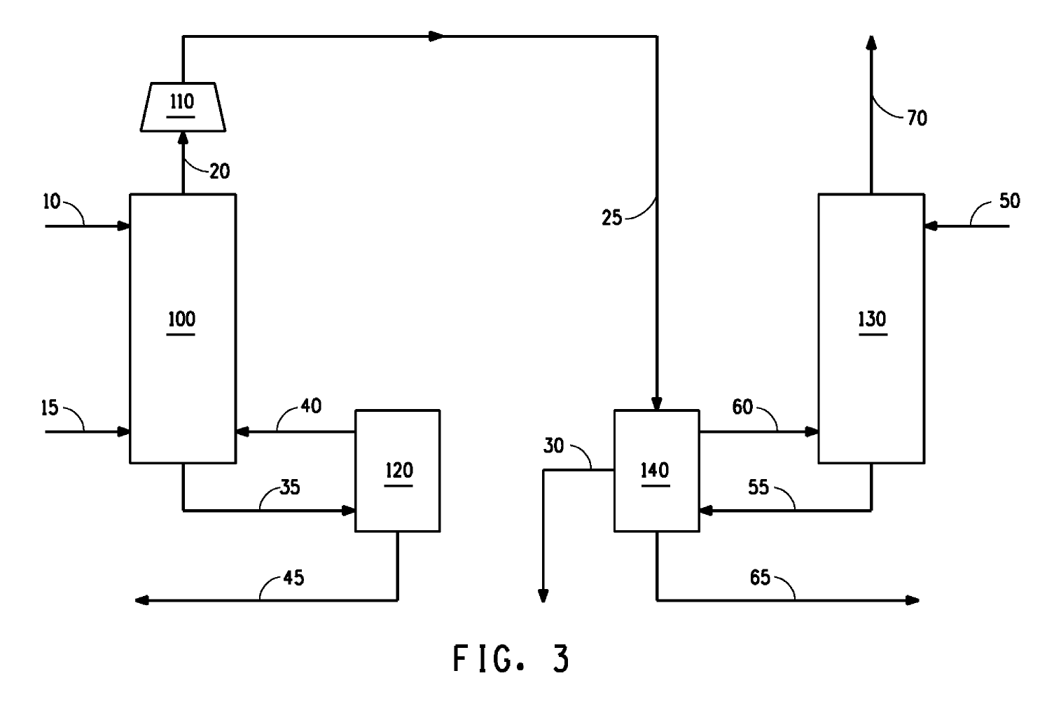

As illustrated in FIG. 3, in certain embodiments vapor recompression from a first distillation column may be used to provide heat energy for a reboiler of a second distillation column. In FIG. 3, a liquid feed steam 10 comprising butanol and water and may be top fed to a distillation column 100, wherein the distillation column may comprise a reboiler 120. A vapor stream 15 comprising water may be fed to the bottom of the column to provide additional heat. The overheads vapor product 20 comprising butanol and water may be produced at a first temperature T₁ and first pressure P₁. The overheads stream may be compressed using a compressor 110, which may be powered by electricity. The product of the compression may comprise a butanol and water vapor stream 25 at a second temperature T₂ and second pressure P₂, operated such that P₂ is greater than P₁. In this embodiment, the resulting vapor stream 25 may be directed to heat exchanger 140, which is a reboiler for distillation column 130. The vapors may be partially condensed at a third temperature T₃, producing stream 30, to provide heat to boil stream 55 comprising butanol and water at a fourth temperature T₄, operated such that T₄ is at least 5° C. less than T₃. Distillation column 130 may be fed by a liquid stream 50, wherein the liquid stream may comprise butanol and water to produce a overheads vapor stream 70 comprising butanol and water and a bottoms product stream 65 comprising water.

In some embodiments, vapor from a distillation column may be recompressed to provide heat energy to a vaporizer that feeds vapor to the bottom of a second distillation column. The vaporizer may be differentiated from a reboiler in that it may receive liquid feed from a process location other than the bottoms of the second distillation column. For example, the liquid feed to the vaporizer may be sourced from the bottoms of a third distillation column or may be process water. The vaporizer may be a boiler or may be an evaporator body used to produce process vapor.

### Recombinant Microorganisms

While not wishing to be bound by theory, it is believed that the processes described herein are useful in conjunction with any alcohol producing microorganism, particularly recombinant microorganisms which produce alcohol.

Recombinant microorganisms which produce alcohol are also known in the art (e.g., Ohta et al., Appl. Environ. Microbiol. 57:893-900 (1991); Underwood et al., Appl. Envrion. Microbiol. 68:1071-81 (2002); Shen and Liao, Metab. Eng. 10:312-20 (2008); Hahnai et al., Appl. Environ. 73:7814-8 (2007); U.S. Pat. No. 5,514,583; U.S. Pat. No. 5,712,133; International Publication No. WO 1995/028476; Feldmann et al., Appl. Microbiol. Biotechnol. 38:354-61 (1992); Zhang et al., Science 267:240-3 (1995); U.S. Patent Publication No. 2007/0031918A1; U.S. Pat. No. 7,223,575; U.S. Pat. No. 7,741,119; U.S. Patent Publication No. 2009/0203099A1; U.S. Patent Publication No. 2009/0246846A1; and International Publication No. WO 2010/075241, which are herein incorporated by reference).

For example, the metabolic pathways of microorganisms may be genetically modified to produce butanol. These pathways may also be modified to reduce or eliminate undesired metabolites, and thereby improve yield of the product alcohol. The production of butanol by a microorganism is disclosed, for example, in U.S. Pat. Nos. 7,851,188; 7,993,889; 8,178,328, 8,206,970; U.S. Patent Application Publication Nos. 2007/0292927; 2008/0182308; 2008/0274525; 2009/0305363; 2009/0305370; 2011/0250610; 2011/0313206; 2011/0111472; 2012/0258873; and 2013/0071898, the entire contents of each are herein incorporated by reference. In some embodiments, microorganisms comprise a butanol biosynthetic pathway or a biosynthetic pathway for a butanol isomer such as 1-butanol, 2-butanol, or isobutanol. In some embodiments, the biosynthetic pathway converts pyruvate to a fermentative product. In some embodiments, the biosynthetic pathway converts pyruvate as well as amino acids to a fermentative product. In certain embodiments, at least one, at least two, at least three, at least four, or at least five polypeptides catalyzing substrate to product conversions in the butanol biosynthetic pathway are encoded by heterologous polynucleotides in the microorganism. In certain embodiments, all the polypeptides catalyzing substrate to product conversions of the butanol biosynthetic pathway are encoded by heterologous polynucleotides in the microorganism. In will be appreciated that microorganisms comprising a butanol biosynthetic pathway may further comprise one or more additional genetic modifications as disclosed in U.S. Patent Application Publication No. 2013/0071898, which is herein incorporated by reference in its entirety.

The recombinant microorganisms of the present invention are engineered to produce 1-butanol, 2-butanol, or isobutanol without undergoing an acetone, butanol, ethanol (ABE) fermentation. The recombinant microorganisms fail to produce acetone or may be engineered to not produce acetone. Thus, in certain embodiments, the product alcohol stream from the fermentation comprises no acetone. The recombinant microorganisms fail to produce or may be engineered to not produce ethanol. Thus, in certain embodiments, the product alcohol stream produced from the fermentation process comprises no ethanol. In certain embodiments, the recombinant microorganism produce trace amounts of ethanol. By way of an example, a recombinant microorganism that produces trace amounts of ethanol may have a butanol to ethanol ratio of at least 10 to 1, whereas an ABE fermentation produces a butanol to ethanol ratio of 6 to 1. Thus, in certain embodiments, the product alcohol stream produced from the fermentation process can comprise ethanol, wherein the butanol to ethanol ratio is at least 10 to 1.

In some embodiments, the microorganism may be bacteria, cyanobacteria, filamentous fungi, or yeasts. Suitable microorganisms capable of producing product alcohol (e.g., butanol) via a biosynthetic pathway include a member of the genera Clostridium, Zymomonas, Escherichia, Salmonella, Serratia, Erwinia, Klebsiella, Shigella, Rhodococcus, Pseudomonas, Bacillus, Lactobacillus, Enterococcus, Alcaligenes, Paenibacillus, Arthrobacter, Corynebacterium, Brevibacterium, Schizosaccharomyces, Kluveromyces, Yarrowia, Pichia, Zygosaccharomyces, Debaryomyces, Candida, Brettanomyces, Pachysolen, Hansenula, Issatchenkia, Trichosporon, Yamadazyma, or Saccharomyces. In one embodiment, recombinant microorganisms may be selected from the group consisting of Escherichia coli, Alcaligenes eutrophus, Bacillus lichenifonnis, Paenibacillus macerans, Rhodocuccus erythropolis, Pseudomonas putida, Lactobacillus plantarum, Enterococcus faecium, Enterococcus gallinarium, Enterococcus faecalis, Bacillus subtilis, Candida sonorensis, Candida methanosorbosa, Kluyveromyces lactis, Kluyveromyces marxianus, Kluveromyces thermotolerans, Issatchenkia orientalis, Debaryomyces hansenii, and Saccharomyces cerevisiae. In one embodiment, the genetically modified microorganism is yeast. In one embodiment, the genetically modified microorganism is a crabtree-positive yeast selected from Saccharomyces, Zygosaccharomyces, Schizosaccharomyces, Dekkera, Torulopsis, Brettanomyces, and some species of Candida. Species of crabtree-positive yeast include, but are not limited to, Saccharomyces cerevisiae, Saccharomyces kluyveri, Schizosaccharomyces pombe, Saccharomyces bayanus, Saccharomyces mikitae, Saccharomyces paradoxus, Saccharomyces uvarum, Saccharomyces castelli, Zygosaccharomyces rouxii, Zygosaccharomyces bailli, and Candida glabrata.

In some embodiments, the host cell is Saccharomyces cerevisiae. Saccharomyces cerevisiae are known in the art and are available from a variety of sources including, but not limited to, American Type Culture Collection (Rockville, Md.), Centraalbureau voor Schimmelcultures (CBS) Fungal Biodiversity Centre, LeSaffre, Gert Strand AB, Ferm Solutions, North American Bioproducts, Martrex, and Lallemand. S. cerevisiae include, but are not limited to, BY4741, CEN.PK 113-7D, Ethanol Red® yeast, Ferm Pro™ yeast, Bio-Ferm® XR yeast, Gert Strand Prestige Batch Turbo alcohol yeast, Gert Strand Pot Distillers yeast, Gert Strand Distillers Turbo yeast, FerMax™ Green yeast, FerMax™ Gold yeast, Thermosacc® yeast, BG-1, PE-2, CAT-1, CBS7959, CBS7960, and CBS7961.

In some embodiments, the microorganism may be immobilized or encapsulated. For example, the microorganism may be immobilized or encapsulated using alginate, calcium alginate, or polyacrylamide gels, or through the induction of biofilm formation onto a variety of high surface area support matrices such as diatomite, celite, diatomaceous earth, silica gels, plastics, or resins. In some embodiments, ISPR may be used in combination with immobilized or encapsulated microorganisms. This combination may improve productivity such as specific volumetric productivity, metabolic rate, product alcohol yields, tolerance to product alcohol. In addition, immobilization and encapsulation may minimize the effects of the process conditions such as shearing on the microorganisms.

Biosynthetic pathways for the production of isobutanol that may be used include those as described by Donaldson et al. in U.S. Pat. No. 7,851,188; U.S. Pat. No. 7,993,388; and International Publication No. WO 2007/050671, which are incorporated herein by reference. In one embodiment, the isobutanol biosynthetic pathway comprises the following substrate to product conversions:

a) pyruvate to acetolactate, which may be catalyzed, for example, by acetolactate synthase;

b) acetolactate to 2,3-dihydroxyisovalerate, which may be catalyzed, for example, by ketol-acid reductoisomerase;

c) 2,3-dihydroxyisovalerate to α-ketoisovalerate, which may be catalyzed, for example, by dihydroxyacid dehydratase;

d) α-ketoisovalerate to isobutyraldehyde, which may be catalyzed, for example, by a branched-chain α-keto acid decarboxylase; and,

e) isobutyraldehyde to isobutanol, which may be catalyzed, for example, by a branched-chain alcohol dehydrogenase.

In another embodiment, the isobutanol biosynthetic pathway comprises the following substrate to product conversions:

a) pyruvate to acetolactate, which may be catalyzed, for example, by acetolactate synthase;

b) acetolactate to 2,3-dihydroxyisovalerate, which may be catalyzed, for example, by ketol-acid reductoisomerase;

c) 2,3-dihydroxyisovalerate to α-ketoisovalerate, which may be catalyzed, for example, by dihydroxyacid dehydratase;

d) α-ketoisovalerate to valine, which may be catalyzed, for example, by transaminase or valine dehydrogenase;

e) valine to isobutylamine, which may be catalyzed, for example, by valine decarboxylase;

f) isobutylamine to isobutyraldehyde, which may be catalyzed by, for example, omega transaminase; and,

g) isobutyraldehyde to isobutanol, which may be catalyzed, for example, by a branched-chain alcohol dehydrogenase.

In another embodiment, the isobutanol biosynthetic pathway comprises the following substrate to product conversions:

a) pyruvate to acetolactate, which may be catalyzed, for example, by acetolactate synthase;

b) acetolactate to 2,3-dihydroxyisovalerate, which may be catalyzed, for example, by ketol-acid reductoisomerase;

c) 2,3-dihydroxyisovalerate to α-ketoisovalerate, which may be catalyzed, for example, by dihydroxy dehydratase;

d) α-ketoisovalerate to isobutyryl-CoA, which may be catalyzed, for example, by branched-chain keto acid dehydrogenase;

e) isobutyryl-CoA to isobutyraldehyde, which may be catalyzed, for example, by acylating aldehyde dehydrogenase; and,

f) isobutyraldehyde to isobutanol, which may be catalyzed, for example, by a branched-chain alcohol dehydrogenase.

Biosynthetic pathways for the production of 1-butanol that may be used include those described in U.S. Patent Application Publication No. 2008/0182308 and WO2007/041269, which are incorporated herein by reference. In one embodiment, the 1-butanol biosynthetic pathway comprises the following substrate to product conversions:

a) acetyl-CoA to acetoacetyl-CoA, which may be catalyzed, for example, by acetyl-CoA acetyltransferase;

b) acetoacetyl-CoA to 3-hydroxybutyryl-CoA, which may be catalyzed, for example, by 3-hydroxybutyryl-CoA dehydrogenase;

c) 3-hydroxybutyryl-CoA to crotonyl-CoA, which may be catalyzed, for example, by crotonase;

d) crotonyl-CoA to butyryl-CoA, which may be catalyzed, for example, by butyryl-CoA dehydrogenase;

e) butyryl-CoA to butyraldehyde, which may be catalyzed, for example, by butyraldehyde dehydrogenase; and,

f) butyraldehyde to 1-butanol, which may be catalyzed, for example, by butanol dehydrogenase.

Biosynthetic pathways for the production of 2-butanol that may be used include those described by Donaldson et al. in U.S. Pat. No. 8,206,970; U.S. Patent Application Publication Nos. 2007/0292927 and 2009/0155870; International Publication Nos. WO 2007/130518 and WO 2007/130521, all of which are incorporated herein by reference. In one embodiment, the 2-butanol biosynthetic pathway comprises the following substrate to product conversions:

a) pyruvate to alpha-acetolactate, which may be catalyzed, for example, by acetolactate synthase;

b) alpha-acetolactate to acetoin, which may be catalyzed, for example, by acetolactate decarboxylase;

c) acetoin to 3-amino-2-butanol, which may be catalyzed, for example, acetonin aminase;

d) 3-amino-2-butanol to 3-amino-2-butanol phosphate, which may be catalyzed, for example, by aminobutanol kinase;

e) 3-amino-2-butanol phosphate to 2-butanone, which may be catalyzed, for example, by aminobutanol phosphate phosphorylase; and,

f) 2-butanone to 2-butanol, which may be catalyzed, for example, by butanol dehydrogenase.

In another embodiment, the 2-butanol biosynthetic pathway comprises the following substrate to product conversions:

a) pyruvate to alpha-acetolactate, which may be catalyzed, for example, by acetolactate synthase;

b) alpha-acetolactate to acetoin, which may be catalyzed, for example, by acetolactate decarboxylase;

c) acetoin to 2,3-butanediol, which may be catalyzed, for example, by butanediol dehydrogenase;

d) 2,3-butanediol to 2-butanone, which may be catalyzed, for example, by dial dehydratase; and,

e) 2-butanone to 2-butanol, which may be catalyzed, for example, by butanol dehydrogenase.

Biosynthetic pathways for the production of 2-butanone that may be used include those described in U.S. Pat. No. 8,206,970 and U.S. Patent Application Publication Nos. 2007/0292927 and 2009/0155870, which are incorporated herein by reference. In one embodiment, the 2-butanone biosynthetic pathway comprises the following substrate to product conversions:

a) pyruvate to alpha-acetolactate, which may be catalyzed, for example, by acetolactate synthase;

b) alpha-acetolactate to acetoin, which may be catalyzed, for example, by acetolactate decarboxylase;

c) acetoin to 3-amino-2-butanol, which may be catalyzed, for example, acetonin aminase;

d) 3-amino-2-butanol to 3-amino-2-butanol phosphate, which may be catalyzed, for example, by aminobutanol kinase; and,

e) 3-amino-2-butanol phosphate to 2-butanone, which may be catalyzed, for example, by aminobutanol phosphate phosphorylase.

In another embodiment, the 2-butanone biosynthetic pathway comprises the following substrate to product conversions:

a) pyruvate to alpha-acetolactate, which may be catalyzed, for example, by acetolactate synthase;

b) alpha-acetolactate to acetoin which may be catalyzed, for example, by acetolactate decarboxylase;

c) acetoin to 2,3-butanediol, which may be catalyzed, for example, by butanediol dehydrogenase;

d) 2,3-butanediol to 2-butanone, which may be catalyzed, for example, by diol dehydratase.

It will be appreciated that host cells comprising an isobutanol biosynthetic pathway as provided herein may further comprise one or more additional modifications. U.S. Patent Application Publication No. 2009/0305363 (incorporated by reference) discloses increased conversion of pyruvate to acetolactate by engineering yeast for expression of a cytosol-localized acetolactate synthase and substantial elimination of pyruvate decarboxylase activity. In some embodiments, the host cells comprise modifications to reduce glycerol-3-phosphate dehydrogenase activity and/or disruption in at least one gene encoding a polypeptide having pyruvate decarboxylase activity or a disruption in at least one gene encoding a regulatory element controlling pyruvate decarboxylase gene expression as described in U.S. Patent Application Publication No. 2009/0305363 (incorporated herein by reference), modifications to a host cell that provide for increased carbon flux through an Entner-Doudoroff Pathway or reducing equivalents balance as described in U.S. Patent Application Publication No. 2010/0120105 (incorporated herein by reference). Other modifications include integration of at least one polynucleotide encoding a polypeptide that catalyzes a step in a pyruvate-utilizing biosynthetic pathway.

Other modifications include at least one deletion, mutation, and/or substitution in an endogenous polynucleotide encoding a polypeptide having acetolactate reductase activity. As used herein, “acetolactate reductase activity” refers to the activity of any polypeptide having the ability to catalyze the conversion of acetolactate to DHMB. Such polypeptides can be determined by methods well known in the art and disclosed herein. As used herein, “DHMB” refers to 2,3-dihydroxy-2-methyl butyrate. DHMB includes “fast DHMB,” which has the 2S, 3S configuration, and “slow DHMB,” which has the 2S, 3R configurate. See Kaneko et al., Phytochemistry 39: 115-120 (1995), which is herein incorporated by reference in its entirety and refers to fast DHMB as anglyceric acid and slow DHMB as tiglyceric acid. In embodiments, the polypeptide having acetolactate reductase activity is YMR226C of Saccharomyces cerevisiae or a homolog thereof

Additional modifications include a deletion, mutation, and/or substitution in an endogenous polynucleotide encoding a polypeptide having aldehyde dehydrogenase and/or aldehyde oxidase activity. As used herein, “aldehyde dehydrogenase activity” refers to any polypeptide having a biological function of an aldehyde dehydrogenase. Such polypeptides include a polypeptide that catalyzes the oxidation (dehydrogenation) of aldehydes. Such polypeptides include a polypeptide that catalyzes the conversion of isobutyraldehyde to isobutyric acid. Such polypeptides also include a polypeptide that corresponds to Enzyme Commission Numbers EC 1.2.1.3, EC 1.2.1.4 or EC 1.2.1.5. Such polypeptides can be determined by methods well known in the art and disclosed herein. As used herein, “aldehyde oxidase activity” refers to any polypeptide having a biological function of an aldehyde oxidase. Such polypeptides include a polypeptide that catalyzes production of carboxylic acids from aldehydes. Such polypeptides include a polypeptide that catalyzes the conversion of isobutyraldehyde to isobutyric acid. Such polypeptides also include a polypeptide that corresponds to Enzyme Commission Number EC 1.2.3.1. Such polypeptides can be determined by methods well known in the art and disclosed herein. In some embodiments, the polypeptide having aldehyde dehydrogenase activity is ALD6 from Saccharomyces cerevisiae or a homolog thereof

A genetic modification which has the effect of reducing glucose repression wherein the yeast production host cell is pdc—is described in U.S. Patent Application Publication No. 2011/0124060, incorporated herein by reference. In some embodiments, the pyruvate decarboxylase that is deleted or down-regulated is selected from the group consisting of: PDC1, PDC5, PDC6, and combinations thereof. In some embodiments, the pyruvate decarboxylase is selected from PDC1 pyruvate decarboxylase from Saccharomyces cerevisiae, PDC5 pyruvate decarboxylase from Saccharomyces cerevisiae, PDC6 pyruvate decarboxylase from Saccharomyces cerevisiae, pyruvate decarboxylase from Candida glabrata, PDC1 pyruvate decarboxylase from Pichia stipites, PDC2 pyruvate decarboxylase from Pichia stipites, pyruvate decarboxylase from Kluveromyces lactis, pyruvate decarboxylase from Yarrowia lipolytica, pyruvate decarboxylase from Schizosaccharomyces pombe, and pyruvate decarboxylase from Zygosaccharomyces rouxii. In some embodiments, host cells contain a deletion or down-regulation of a polynucleotide encoding a polypeptide that catalyzes the conversion of glyceraldehyde-3-phosphate to glycerate 1,3, bisphosphate. In some embodiments, the enzyme that catalyzes this reaction is glyceraldehyde-3-phosphate dehydrogenase.

WIPO publication number WO 2001/103300 discloses recombinant host cells comprising (a) at least one heterologous polynucleotide encoding a polypeptide having dihydroxy-acid dehydratase activity; and (b)(i) at least one deletion, mutation, and/or substitution in an endogenous gene encoding a polypeptide affecting Fe—S cluster biosynthesis; and/or (ii) at least one heterologous polynucleotide encoding a polypeptide affecting Fe—S cluster biosynthesis. In embodiments, the polypeptide affecting Fe—S cluster biosynthesis is encoded by AFT1, AFT2, FRA2, GRX3, or CCC1. In embodiments, the polypeptide affecting Fe—S cluster biosynthesis is constitutive mutant AFT1 L99A, AFT1 L102A, AFT1 C291F, or AFT1 C293F.

Additionally, host cells may comprise heterologous polynucleotides encoding a polypeptide with phosphoketolase activity and/or a heterologous polynucleotide encoding a polypeptide with phosphotransacetylase activity.

In some embodiments, any particular nucleic acid molecule or polypeptide may be at least 80%, 85%, 90%, 95%, 96%, 97%, 98%, or 99% identical to a nucleotide sequence or polypeptide sequence described herein. The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those disclosed in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Methods to determine identity are designed to give the best match between the sequences tested. Methods to determine identity and similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” corresponding to the alignment method labeled Clustal V (disclosed by Higgins and Sharp, CABIOS. 5:151-153, 1989; Higgins, et al., Comput. Appl. Biosci. 8:189-191, 1992) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. Additionally the “Clustal W method of alignment” is available and corresponds to the alignment method labeled Clustal W (described by Higgins and Sharp, CABIOS. 5:151-153, 1989; Higgins, et al., Comput. Appl. Biosci. 8:189-191, 1992) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

Standard recombinant DNA and molecular cloning techniques are well known in the art and are described by Sambrook, et al. (Sambrook, J., Fritsch, E. F. and Maniatis, T. (Molecular Cloning: A Laboratory Manual; Cold Spring Harbor Laboratory Press, Cold Spring Harbor, 1989, here in referred to as Maniatis) and by Ausubel, et al. (Ausubel, et al., Current Protocols in Molecular Biology, pub. by Greene Publishing Assoc. and Wiley-Interscience, 1987). Examples of methods to construct microorganisms that comprise a butanol biosynthetic pathway are disclosed, for example, in U.S. Pat. No. 7,851,188, and U.S. Patent Application Publication Nos. 2007/0092957; 2007/0259410; 2007/0292927; 2008/0182308; 2008/0274525; 2009/0155870; 2009/0305363; and 2009/0305370, the entire contents of each are herein incorporated by reference.

### Organic Extractants

Extractants useful in the methods described herein are water immiscible organic solvents. Suitable organic extractants include those that have one or more or all of the following characteristics: (i) biocompatible to the butanol-producing microorganisms such as, for example, the recombinant microorganisms described herein, (ii) substantially immiscible with the fermentation medium, (iii) a high partition coefficient for the extraction of butanol, (iv) have a low partition coefficient for the extraction of nutrients, (v) a low tendency to form emulsions with the fermentation medium, and (vi) low cost and nonhazardous. Suitable organic extractants for use in the methods disclosed herein may comprise C₇ to C₂₂ fatty alcohols, C₇ to C₂₂ fatty acids, esters of C₇ to C₂₂ fatty acids, C₇ to C₂₂ fatty aldehydes, or mixtures thereof. As used herein, the term “mixtures thereof” encompasses both mixtures within and mixtures between these group members, for example, mixtures within C₇ to C₂₂ fatty alcohols, and also mixtures between C₇ to C₂₂ fatty alcohols and C₇ to C₂₂ fatty acids.

Suitable organic extractants may comprise oleyl alcohol (CAS No. 143-28-2), behenyl alcohol (CAS No. 661-19-8), cetyl alcohol (CAS No. 36653-82-4), lauryl alcohol, also referred to as 1-dodecanol (CAS No. 112-53-8), myristyl alcohol (112-72-1), stearyl alcohol (CAS No. 112-92-5), 1-undecanol (CAS No. 112-42-5), oleic acid (CAS No. 112-80-1), lauric acid (CAS No. 143-07-7), myristic acid (CAS No. 544-63-8), stearic acid (CAS No. 57-11-4), methyl myristate (CAS No. 124-10-7), methyl oleate (CAS No. 112-62-9), undecanal (CAS No. 112-44-7), lauric aldehyde (CAS No. 112-54-9), 20-methylundecanal (CAS No. 110-41-8), or mixtures thereof. These organic extractants are available commercially from various sources, such as Sigma-Aldrich (St. Louis, Mo.), in various grades, many of which may be suitable for use in extractive fermentation to produce or recover butanol. Technical grades contain a mixture of compounds, including the desired component and higher and lower fatty components. For example, one commercially available technical grade oleyl alcohol contains about 65% oleyl alcohol and a mixture of higher and lower fatty alcohols.

In certain embodiments, the extractant can be one or more of the following fatty acids: azaleic, capric, caprylic, castor, coconut (i.e., as a naturally-occurring combination of fatty acids, including lauric, myrisitic, palmitic, caprylic, capric, stearic, caproic, arachidic, oleic, and linoleic, for example), dimer, isostearic, lauric, linseed, myristic, oleic, olive, palm oil, palmitic, palm kernel, peanut, pelargonic, ricinoleic, sebacic, soya, stearic acid, tall oil, tallow, #12 hydroxy stearic, or any seed oil. In some embodiments, the extractant can be one or more of diacids, azelaic, dimer and sebacic acid. Thus, in some embodiments, the extractant can be a mixture of two or more different fatty acids. In some embodiments, the extractant can be a fatty acid derived from chemical or enzymatic hydrolysis of glycerides derived from native oil. For example, in some embodiments, the extractant can be free fatty acids obtained by enzymatic hydrolysis of native oil such as biomass lipids. In some embodiments, the extractant can be a fatty acid extractant selected from the group consisting of fatty acids, fatty alcohols, fatty amides, fatty acid methyl esters, lower alcohol esters of fatty acids, fatty acid glycol esters, hydroxylated triglycerides, and mixtures thereof, obtained from chemical conversion of native oil such as biomass lipids as described in U.S. Patent Publication No. 2011/0312044. In such embodiments, the biomass lipids for producing extractant can be from a same or different biomass source from which feedstock is obtained. For example, in some embodiments, the biomass lipids for producing extractant can be derived from soya, whereas the biomass source of feedstock is corn. Any possible combination of different biomass sources for extractant versus feedstock can be used, as should be apparent to one of skill in the art. In some embodiments, additional extractant includes fatty acids derived from corn oil (COFA).

One of reasonable skill in the art can appreciate that it may be advantageous to use a mixture of the organic extractants. For example, solvent mixtures may be used to increase the partition coefficient of the product. Additionally, solvent mixtures may be used to adjust and optimize physical characteristics of the solvent, such as the density, boiling point, surface tension, and viscosity.

### Growth for Production

Recombinant host cells disclosed herein are grown in fermentation media which contains suitable carbon substrates. Additional carbon substrates may include, but are not limited to, monosaccharides such as fructose, oligosaccharides such as lactose, maltose, galactose, or sucrose, polysaccharides such as starch or cellulose or mixtures thereof and unpurified mixtures from renewable feedstocks such as cheese whey permeate, cornsteep liquor, sugar beet molasses, and barley malt. Other carbon substrates may include ethanol, lactate, succinate, or glycerol.

Additionally the carbon substrate may also be one-carbon substrates such as carbon dioxide, or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeasts are known to utilize the carbon from methylamine to form trehalose or glycerol (Bellion et al., Microb. Growth C1 Compd., [Int. Symp.], 7th (1993), 415-32, Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, in some embodiments, the carbon substrates are glucose, fructose, and sucrose, or mixtures of these with C5 sugars such as xylose and/or arabinose for yeasts cells modified to use C5 sugars. Sucrose may be derived from renewable sugar sources such as sugar cane, sugar beets, cassava, sweet sorghum, and mixtures thereof. Glucose and dextrose may be derived from renewable grain sources through saccharification of starch based feedstocks including grains such as corn, wheat, rye, barley, oats, and mixtures thereof. In addition, fermentable sugars may be derived from renewable cellulosic or lignocellulosic biomass through processes of pretreatment and saccharification, as described, for example, in U.S. Patent Application Publication No. 2007/0031918 A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof

In addition to an appropriate carbon source, fermentation media must contain suitable minerals, salts, cofactors, buffers and other components, known to those skilled in the art, suitable for the growth of the cultures and promotion of an enzymatic pathway described herein.

### Culture Conditions

Typically cells are grown at a temperature in the range of about 20° C. to about 40° C. in an appropriate medium. Suitable growth media in the present invention are common commercially prepared media such as Luria Bertani (LB) broth, Sabouraud Dextrose (SD) broth, Yeast Medium (YM) broth, or broth that includes yeast nitrogen base, ammonium sulfate, and dextrose (as the carbon/energy source) or YPD Medium, a blend of peptone, yeast extract, and dextrose in optimal proportions for growing most Saccharomyces cerevisiae strains. Other defined or synthetic growth media may also be used, and the appropriate medium for growth of the particular microorganism will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation are between about pH 5.0 to about pH 9.0. In one embodiment, about pH 6.0 to about pH 8.0 is used for the initial condition. Suitable pH ranges for the fermentation of yeast are typically between about pH 3.0 to about pH 9.0. In one embodiment, about pH 5.0 to about pH 8.0 is used for the initial condition. Suitable pH ranges for the fermentation of other microorganisms are between about pH 3.0 to about pH 7.5. In one embodiment, about pH 4.5 to about pH 6.5 is used for the initial condition.

Fermentations may be performed under aerobic or anaerobic conditions. In one embodiment, anaerobic or microaerobic conditions are used for fermentations.

### Industrial Batch and Continuous Fermentations

Butanol, or other products, may be produced using a batch method of fermentation. A classical batch fermentation is a closed system where the composition of the medium is set at the beginning of the fermentation and not subject to artificial alterations during the fermentation. A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Batch and fed-batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

Butanol, or other products, may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology and a variety of methods are detailed by Brock, supra.

It is contemplated that the production of butanol, or other products, may be practiced using batch, fed-batch or continuous processes and that any known mode of fermentation would be suitable. Additionally, it is contemplated that cells may be immobilized on a substrate as whole cell catalysts and subjected to fermentation conditions for butanol production.

Methods for Butanol Isolation from the Fermentation Medium

Bioproduced butanol may be isolated from the fermentation medium using methods known in the art for ABE fermentations (see, e.g., Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. The butanol may be isolated from the fermentation medium using methods such as distillation, azeotropic distillation, liquid-liquid extraction, adsorption, gas stripping, membrane evaporation, or pervaporation.

Because butanol forms a low boiling point, azeotropic mixture with water, distillation can be used to separate the mixture up to its azeotropic composition. Distillation may be used in combination with the processes described herein to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer (see, e.g., Doherty and Malone, Conceptual Design of Distillation Systems, McGraw Hill, New York, 2001).

The butanol-water mixture forms a heterogeneous azeotrope so that distillation may be used in combination with decantation to isolate and purify the isobutanol. In this method, the butanol containing fermentation broth is distilled to near the azeotropic composition. Then, the azeotropic mixture is condensed, and the butanol is separated from the fermentation medium by decantation. The decanted aqueous phase may be returned to the first distillation column as reflux or to a separate stripping column. The butanol-rich decanted organic phase may be further purified by distillation in a second distillation column.

The butanol can also be isolated from the fermentation medium using liquid-liquid extraction in combination with distillation. In this method, the butanol is extracted from the fermentation broth using liquid-liquid extraction with a suitable solvent. The butanol-containing organic phase is then distilled to separate the butanol from the solvent.

Distillation in combination with adsorption can also be used to isolate butanol from the fermentation medium. In this method, the fermentation broth containing the butanol is distilled to near the azeotropic composition and then the remaining water is removed by use of an adsorbent, such as molecular sieves (Aden et al., Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the butanol from the fermentation medium. In this method, the fermentation broth containing the butanol is distilled to near the azeotropic composition, and then the remaining water is removed by pervaporation through a hydrophilic membrane (Guo et al., J. Membr. Sci. 245, 199-210 (2004)).

In situ product removal (ISPR) (also referred to as extractive fermentation) can be used to remove butanol (or other fermentative alcohol) from the fermentation vessel as it is produced, thereby allowing the microorganism to produce butanol at high yields. One method for ISPR for removing fermentative alcohol that has been described in the art is liquid-liquid extraction. In general, with regard to butanol fermentation, for example, the fermentation medium, which includes the microorganism, is contacted with an organic extractant at a time before the butanol concentration reaches a toxic level. The organic extractant and the fermentation medium form a biphasic mixture. The butanol partitions into the organic extractant phase, decreasing the concentration in the aqueous phase containing the microorganism, thereby limiting the exposure of the microorganism to the inhibitory butanol.

Liquid-liquid extraction can be performed, for example, according to the processes described in U.S. Patent Appl. Pub. No. 2009/0305370, the disclosure of which is hereby incorporated in its entirety. U.S. Patent Appl. Pub. No. 2009/0305370 describes methods for producing and recovering butanol from a fermentation broth using liquid-liquid extraction, the methods comprising the step of contacting the fermentation broth with a water immiscible extractant to form a two-phase mixture comprising an aqueous phase and an organic phase. Typically, the extractant can be an organic extractant selected from the group consisting of saturated, mono-unsaturated, poly-unsaturated (and mixtures thereof) C₁₂ to C₂₂ fatty alcohols, C₁₂ to C₂₂ fatty acids, esters of C₁₂ to C₂₂ fatty acids, C₁₂ to C₂₂ fatty aldehydes, and mixtures thereof. The extractant(s) for ISPR can be non-alcohol extractants. The ISPR extractant can be an exogenous organic extractant such as oleyl alcohol, behenyl alcohol, cetyl alcohol, lauryl alcohol, myristyl alcohol, stearyl alcohol, 1-undecanol, oleic acid, lauric acid, myristic acid, stearic acid, methyl myristate, methyl oleate, undecanal, lauric aldehyde, 20-methylundecanal, and mixtures thereof

In some embodiments, an alcohol ester can be formed by contacting the alcohol in a fermentation medium with an organic acid (e.g., fatty acids) and a catalyst capable of esterifying the alcohol with the organic acid. In such embodiments, the organic acid can serve as an ISPR extractant into which the alcohol esters partition. The organic acid can be supplied to the fermentation vessel and/or derived from the biomass supplying fermentable carbon fed to the fermentation vessel. Lipids present in the feedstock can be catalytically hydrolyzed to organic acid, and the same catalyst (e.g., enzymes) can esterify the organic acid with the alcohol. Carboxylic acids that are produced during the fermentation can additionally be esterified with the alcohol produced by the same or a different catalyst. The catalyst can be supplied to the feedstock prior to fermentation, or can be supplied to the fermentation vessel before or contemporaneously with the supplying of the feedstock. When the catalyst is supplied to the fermentation vessel, alcohol esters can be obtained by hydrolysis of the lipids into organic acid and substantially simultaneous esterification of the organic acid with butanol present in the fermentation vessel. Organic acid and/or native oil not derived from the feedstock can also be fed to the fermentation vessel, with the native oil being hydrolyzed into organic acid. Any organic acid not esterified with the alcohol can serve as part of the ISPR extractant. The extractant containing alcohol esters can be separated from the fermentation medium, and the alcohol can be recovered from the extractant. The extractant can be recycled to the fermentation vessel. Thus, in the case of butanol production, for example, the conversion of the butanol to an ester reduces the free butanol concentration in the fermentation medium, shielding the microorganism from the toxic effect of increasing butanol concentration. In addition, unfractionated grain can be used as feedstock without separation of lipids therein, since the lipids can be catalytically hydrolyzed to organic acid, thereby decreasing the rate of build-up of lipids in the ISPR extractant.

In situ product removal can be carried out in a batch mode or a continuous mode. In a continuous mode of in situ product removal, product is continually removed from the reactor. In a batchwise mode of in situ product removal, a volume of organic extractant is added to the fermentation vessel and the extractant is not removed during the process. For in situ product removal, the organic extractant can contact the fermentation medium at the start of the fermentation forming a biphasic fermentation medium. Alternatively, the organic extractant can contact the fermentation medium after the microorganism has achieved a desired amount of growth, which can be determined by measuring the optical density of the culture. Further, the organic extractant can contact the fermentation medium at a time at which the product alcohol level in the fermentation medium reaches a preselected level. Accordingly, in situ product removal can be carried out whereby butanol is contacted with extractant in the fermenter or such contact may occur downstream of the fermenter. In the case of butanol production according to some embodiments of the present invention, the organic acid extractant can contact the fermentation medium at a time before the butanol concentration reaches a toxic level, so as to esterify the butanol with the organic acid to produce butanol esters and consequently reduce the concentration of butanol in the fermentation vessel. The ester-containing organic phase can then be removed from the fermentation vessel (and separated from the fermentation broth which constitutes the aqueous phase) after a desired effective titer of the butanol esters is achieved. In some embodiments, the ester-containing organic phase is separated from the aqueous phase after fermentation of the available fermentable sugar in the fermentation vessel is substantially complete.

### Ethanol Plant Retrofit

In certain embodiments, a biobutanol production plant can be retrofitted from a bioethanol plant. By way of an example, the equipment and configuration of the equipment in a bioethanol plant may be altered to enable fermentation and purification of biobutanol, with an objective of the conversion to minimize use of capital. Retrofitting the equipment may include both modification of existing equipment and installation of new equipment. To minimize loss of operational flexibility, the conversion of a bioethanol plant to a biobutanol plant may be reversible. Reversibly retrofitting a bioethanol plant to a biobutanol production plant is described in U.S. Patent Publication No. 2013/0309738, the entire contents of which are herein incorporated by reference.

Traditional bioethanol production involves the purification of an end-of-fermentation mixture, called beer, comprising ethanol, water and biomass. This mixture can be top-fed to a beer column. The mixture in the beer column can be contacted with an evaporator steam that is fed to the bottom of the column. The resulting overhead product can be a vapor mixture comprising bioethanol and water. The bottoms product, known as whole stillage, can be a liquid mixture comprising water and biomass, which can be substantially free of bioethanol. In methods of producing bioethanol, the evaporator steam can provide the required energy for the beer column to distill the bioethanol from the water.

In certain embodiments of retrofitting a bioethanol plant for biobutanol production, the beer column can be reused for the recovery of biobutanol from fermentation beer. In certain embodiments, the beer column retrofit includes the addition of a column reboiler. In certain embodiments, operation of the beer column includes the use of at least a portion of the evaporator steam to assist in stripping butanol from the beer. The column overhead product can be a vapor mixture comprising butanol and water. The bottoms product can be a liquid comprising biomass and water, which can be substantially free of biobutanol. In certain embodiments of producing biobutanol from a retrofitted bioethanol facility, the distillation requires additional energy than what is provided by the portion of the evaporator steam. The additional energy can be provided by vapor recompression as described herein. Thus, in certain embodiments, the column overhead product, which can be a vapor mixture comprising butanol and water, can be compressed to an elevated pressure for the recovery of thermal energy using methods as described herein. The recovered thermal energy can be used to heat the column reboiler, or the recovered thermal energy can be used to heat another part of the process (e.g., the recovered thermal energy can be used to heat an extractant stream that can be fed to an extractant column).

The use of extractive ISPR in biobutanol production may introduce another biobutanol containing stream which must be separated for the recovery of biobutanol product. The rich organic extractant can be distilled in an extractant column. A liquid mixture comprising biobutanol, water, and biobutanol-selective organic extractant can be top fed to the extractant column. In certain embodiments, at least a portion of the evaporator steam can be bottom fed to the column to enhance biobutanol stripping ability. In certain embodiments, the column comprises a reboiler. The column overhead product can be a vapor mixture comprising butanol and water. The bottoms product can be a liquid comprising butanol selective organic extractant, water and may contain trace amounts of butanol. In certain embodiments of producing biobutanol from a retrofitted bioethanol facility, the distillation requires additional energy. The additional energy can be provided by vapor recompression as described herein. Thus, in certain embodiments, the column overhead product, which can be a vapor mixture comprising biobutanol and water, can be compressed to an elevated pressure for the recovery of thermal energy using methods as described herein. The recovered thermal energy can be used to heat the column reboiler or the recovered thermal energy can be used to heat another part of the process (e.g., the recovered thermal energy can be used to heat the extractant stream being fed to the column).

While various embodiments of the present invention have been described herein, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the claims and their equivalents.

All publications, patents, and patent applications mentioned in this specification are indicative of the level of those skilled in the art to which this invention pertains, and are herein incorporated by reference to the same extent as if each individual publication, patent, or patent application was specifically and individually indicated to be incorporated by reference.

### EXAMPLES

The following nonlimiting examples will further illustrate the invention. It should be understood that, while the following examples involve isobutanol, other alcohols may be produced without departing from the present invention.

The processes described herein may be demonstrated using computational modeling such as Aspen modeling (see, e.g., U.S. Pat. No. 7,666,282). For example, the commercial modeling software Aspen Plus® (Aspen Technology, Inc., Burlington, Mass.) may be used in conjunction with physical property databases such as DIPPR, available from American Institute of Chemical Engineers, Inc. (New York, N.Y.) to develop an Aspen model for an integrated butanol fermentation, purification, and water management process. This process modeling can perform many fundamental engineering calculations, for example, mass and energy balances, vapor/liquid equilibrium, and reaction rate computations. In order to generate an Aspen model, information input may include, for example, experimental data, water content and composition of feedstock, temperature for mash cooking and flashing, saccharification conditions (e.g., enzyme feed, starch conversion, temperature, pressure), fermentation conditions (e.g., microorganism feed, glucose conversion, temperature, pressure), degassing conditions, solvent columns, pre-flash columns, condensers, evaporators, centrifuges, etc.

### Example 1

### ASPEN Model: Use of Vapor Recompression to Self-Heat a Distillation Column Reboiler

An ASPEN model was developed in which isobutanol was produced in a fermentation vessel by a biocatalyst through the consumption of a fermentable carbon source. During fermentation, isobutanol product was continuously removed by an organic extractant to control the isobutanol concentration in the fermentation vessel. The product of the extraction was a composition comprising a butanol selective organic extract, isobutanol and water. To recover the isobutanol product, the composition was distilled.

In distillation, as illustrated in FIG. 1, a composition comprising a butanol selective organic extractant, isobutanol and water was top-fed to a distillation column. As a supplemental source of heat, a vapor stream comprising water was fed to the bottom of the column, which also assisted in stripping butanol from the top-fed composition. In this example, the column operated at a top pressure, P₁, of 0.45 atm. At this pressure, the thermodynamic dew point temperature of the isobutanol and water mixture created as an overheads vapor product was approximately 77° C. The column bottoms operated at a pressure, P₃, of 0.6 atm. At the column bottoms, the equilibrium temperature of the reboiled mixture, T₄, comprising butanol selective organic extractant, isobutanol and water was 86° C.

To supply heat to the column reboiler, the overheads vapor product was isentropically compressed to a pressure, P₂, of 0.9 atm. At this pressure, the isobutanol and water vapor phase mixture had a condensing temperature of T₃, 91° C. The increase in condensing temperature allowed for the column reboiler to be heated by the energy released from condensing the overheads vapor product. A counter-current heat exchanger could be used as the column reboiler. The column reboiler allowed for the column bottoms liquid to be reboiled into the distillation column using at least a portion of the condensing energy of the overheads vapor product. In this example, if the overheads vapor was not fully condensed, further condensing energy could be used on other heat exchangers. The mass balance of the distillation is shown in Table 1.

TABLE 1
Mass balance of distillation.

| | | | | Pressure | |
|---|---|---|---|---|---|
| Stream | X, butanol | X, Water | X, Extractant | (atm) | Temp. (° C.) |
| 10 | 0.018 | 0.018 | 0.964 | 3 | 78.4 |
| 15 | 0 | 1 | 0 | 0.8 | 94.7 |
| 20 | 0.15 | 0.85 | 0 | 0.45 | 78.2 |
| 25 | 0.15 | 0.85 | 0 | 0.9 | 157.8 |
| 30 | 0.15 | 0.85 | 0 | 0.9 | 90.8 |
| 35 | 0.003 | 0.019 | 0.976 | 0.6 | 86.4 |
| 40 | 0.024 | 0.973 | 0 | 0.6 | 86.4 |

### Example 2

### ASPEN Model: Use of Vapor Recompression to Recover Thermal Energy to Preheat Column Feed Stream

An ASPEN model was developed in which isobutanol was produced in a fermentation vessel by a biocatalyst through the consumption of a fermentable carbon source. During fermentation, isobutanol product was continuously removed by an organic extractant to control the isobutanol concentration in the fermentation vessel. The product of the extraction was a composition comprising a butanol selective organic extract, isobutanol and water. To recover the isobutanol product, the composition was distilled.

An illustration describing Example 2 is shown in FIG. 2. In this example, a liquid composition comprising isobutanol and water, stream 75, was top-fed to a distillation column 160. In this embodiment, distillation column 160 was operated at a pressure such that an overheads vapor comprising isobutanol and water was produced at a pressure, P₁, of 1.85 atm. At this pressure, the vapor would condense at a temperature of about 107° C. As a means by which to recover additional thermal energy, the overhead vapor product was isentropically compressed to a pressure P₂ of 4 atm.

A second distillation column 100 was used to distill a second mixture comprising isobutanol and water, which contained a butanol selective organic extractant. This distillation column operated at a tops pressure of 0.1 atm and a bottoms pressure of 0.25 atm. To assist in the recovery of butanol from column 100, the liquid composition was preheated to a temperature T₄. T₄ was selected such that, upon entering the vacuum-operated distillation column, the composition was flashed to remove butanol.

In this example, the compressed overheads vapor was partially condensed across distillation column 100 preheater 160 at a temperature of about 132° C. Energy transferred to the liquid feed heated stream 10 to a temperature of about 128° C. Remaining vapor in stream 115 was condensed at about 132° C. in a separate heat exchanger to recover additional thermal energy. The mass balance of the distillation is shown in Table 2.

TABLE 2
Mass balance of distillation.

| | | | | Pressure | |
|---|---|---|---|---|---|
| Stream | X, butanol | X, Water | X, Extractant | (atm) | Temp. (° C.) |
| 10 | 0.015 | 0.019 | 0.966 | 3 | 125.2 |
| 70 | 0.015 | 0.019 | 0.966 | 3 | 128 |
| 75 | 0.81 | 0.19 | 0 | 2 | 109 |
| 80 | 0.73 | 0.27 | 0 | 1.85 | 108.4 |
| 85 | 0.73 | 0.27 | 0 | 4 | 156.1 |
| 115 | 0.73 | 0.27 | 0 | 4 | 132.1 |

### Example 3

### ASPEN Model: Use of Vapor Recompression to Provide Heat to Reboiler of Second Distillation Column

An ASPEN model was developed in which isobutanol was produced in a fermentation vessel by a biocatalyst through the consumption of a fermentable carbon source. During fermentation, isobutanol was continuously removed by an organic extractant to control the isobutanol concentration in the fermentation vessel. The product of the extraction was a composition comprising a butanol selective organic extract, isobutanol and water. To recover the isobutanol, the composition was distilled.

Example 3 is illustrated in FIG. 3. In this embodiment, the overheads vapor produced in distillation column 100, comprising vapor phase isobutanol and water at a pressure of 0.45 atm, was isentropically compressed to a pressure of 0.9 atm. The resulting vapor stream, with a condensing temperature of 91° C. was used to supply thermal energy to the reboiler 140 of distillation column 130.

Distillation column 130 was used to remove isobutanol from a liquid mixture 50 comprising isobutanol and water. The operating pressure of the column was selected such that the temperature of the reboiled liquid 60, comprising isobutanol and water, was less than about 86° C. so that the condensing vapor from the overheads of distillation column 100 could be used to supply all of the energy required to the reboiler. In this example, distillation column 130 operated with a bottoms pressure of 0.6 atm resulting in a reboiled vapor stream 60 produced at about 86° C. The compressed vapor 25 was partially condensed across reboiler 140. Remaining vapor in stream 30 was condensed at about 91° C. in a separate heat exchanger to recover additional thermal energy. The mass balance of the distillation is shown in Table 3.

TABLE 3
Mass balance of distillation.

| | | | | Pressure | |
|---|---|---|---|---|---|
| Stream | X, butanol | X, Water | X, Extractant | (atm) | Temp. (° C.) |
| 10 | 0.018 | 0.018 | 0.964 | 3 | 78.4 |
| 15 | 0 | 1 | 0 | 0.8 | 94.7 |
| 20 | 0.15 | 0.85 | 0 | 0.45 | 78.2 |
| 25 | 0.15 | 0.85 | 0 | 0.9 | 157.8 |
| 30 | 0.15 | 0.85 | 0 | 0.9 | 90.8 |
| 50 | 0.013 | 0.976 | 0.011 | 2 | 69.2 |
| 55 | 0 | 0.992 | 0.001 | 0.6 | 86.4 |
| 60 | 0 | 0.999 | 0 | 0.6 | 86.4 |

1. A process comprising:
(a) distilling a butanol and water composition in a distillation unit, whereby the distillation produces a vapor comprising butanol and water at a first pressure P1;
(b) compressing the vapor to a second pressure P2, wherein P2 is greater than P1; and
(c) condensing the vapor to a liquid, whereby condensing the vapor allows for the recovery of thermal energy,
wherein the recovered thermal energy is used as a heat source.

2. The process of claim 1, wherein the butanol is bioproduced.

3. The process of claim 1, wherein the first pressure P1 is in a range from about 0.1 atmosphere (atm) to about 2 atm.

4. (canceled)

5. The process of claim 1, wherein the vapor is compressed by a compressor selected from the group consisting of a centrifugal compressor, an axial compressor, a liquid ring compressor, a reciprocating compressor, a rotary screw compressor and a diaphragm compressor.

6. (canceled)

7. The process of claim 1, wherein the second pressure P2 is in a range from about 0.11 atm to about 10 atm.

8. (canceled)

9. The process of claim 1, wherein the ratio of P2 to P1 is in a range from about 1.1 to about 5.0

10-11. (canceled)

12. The process of claim 1, wherein the distillation unit comprises at least one distillation column.

13. The process of claim 12, wherein the at least one distillation column comprises at least one reboiler.

14. The process of claim 13, wherein the recovered thermal energy is used to heat the at least one reboiler.

15. The process of claim 12, wherein the at least one distillation column comprises a feed, wherein the feed comprises a composition comprising butanol and water.

16. The process of claim 15, wherein the recovered thermal energy is used to heat the feed.

17. A process comprising:
(a) providing a fermentation medium comprising a recombinant microorganism comprising an engineered butanol biosynthetic pathway, whereby the recombinant microorganism produces butanol;
(b) contacting the butanol with an extractant;
(c) distilling the butanol and extractant in a distillation unit, whereby the distillation produces a vapor comprising butanol and water, wherein the vapor is produced at a first pressure P1;
(d) compressing the vapor to a second pressure P2, wherein P2 is greater than P1; and
(e) condensing the vapor to a liquid, whereby condensing the vapor allows for the recovery of thermal energy,
wherein the recovered thermal energy is used as a heat source.

18. The process of claim 17, wherein the engineered butanol biosynthetic pathway is selected from the group consisting of:
(a) a 1-butanol biosynthetic pathway;
(b) a 2-butanol biosynthetic pathway; and
(c) an isobutanol biosynthetic pathway.

19-21. (canceled)

22. The process of claim 17, wherein the extractant comprises C7 to C22 fatty alcohols, esters of C7 to C22 fatty acids, C7 to C22 fatty aldehydes, C7 to C22 fatty amides, or mixtures thereof.

23. The process of claim 17, wherein the extractant comprises oleyl alcohol, behenyl alcohol, cetyl alcohol, lauryl alcohol, myristyl alcohol, stearyl alcohol, oleic acid, lauric acid, myristic acid, stearic acid, octanoic acid, decanoic acid, undecanoic acid, methyl myristate, methyl oleate, 1-nonanol, 1-decanol, 2-undecanol, 1-nonanal, 1-undecanol, undecanal, lauric aldehyde, 2-methylundecanal, oleamide, linoleamide, palmitamide, stearylamide, 2-ethyl-1-hexanol, 2-hexyl-1-decanol, 2-octyl-1-dodecanol, or mixtures thereof.

24. The process of claim 17, wherein the extractant comprises fatty acids derived from a plant source.

25. The process of claim 24, wherein the plant source is corn, sugar cane, wheat, soy, barley, rice, switchgrass, grain, sorghum, whey, or mixtures thereof.

26. The process of claim 17, wherein the first pressure P1 is in a range from about 0.1 atmosphere (atm) to about 2 atm.

27. (canceled)

28. The process of claim 17, wherein the vapor is compressed by a compressor selected from the group consisting of a centrifugal compressor, an axial compressor, a liquid ring compressor, a reciprocating compressor, a rotary screw compressor and a diaphragm compressor.

29. (canceled)

30. The process of claim 17, wherein the second pressure P2 is in a range from about 0.11 atm to about 10 atm.

31. (canceled)

32. The process of claim 17, wherein the ratio of P2 to P1 is in a range from about 1.1 to about 5.0

33-34. (canceled)

35. The process of claim 17, wherein the distillation unit comprises at least one distillation column.

36. The process of claim 35, wherein the at least one distillation column comprises at least one reboiler.

37. The process of claim 36, wherein the recovered thermal energy is used to heat the at least one reboiler.

38. The process of claim 35, wherein the at least one distillation column comprises a feed, wherein the feed comprises a composition comprising butanol and water.

39. The process of claim 38, wherein the recovered thermal energy is used to heat the feed.
